(19) United States
(12) Patent Application Publication
(10) Pub. No.: US 2024/0091041 A1
(43) Pub. Date: Mar. 21, 2024

(54) UPPER EXTREMITY PROSTHETIC DEVICE WITH ENHANCED SPRING DESIGNS

(71) Applicant: Accenture Global Solutions Limited, Dublin (IE)

(72) Inventors: Jung Wook Park, Foster City, CA (US); Eric Michael Gallo, Moretown, VT (US); Lavinia Andreea Danielescu, Seattle, WA (US); Mark Benjamin Greenspan, Walnut Creek, CA (US)

(21) Appl. No.: 18/243,715

(22) Filed: Sep. 8, 2023

Related U.S. Application Data
(60) Provisional application No. 63/407,003, filed on Sep. 15, 2022.

(51) Int. Cl. A61F 5/01

(52) U.S. Cl. CPC A61F 5/013; A61F 2230/0063

(57) ABSTRACT

Springs can provide energy return and have a conductivity that changes in relation to an amount of strain or deformation of the spring. An upper-extremity prosthetic device includes a first coil spring coupled to a first member and a first cantilever spring extending from the first member to a surface adapted to engage with an object. The first coil spring is arranged to absorb energy and to provide energy return in response to movement of the first member. The first coil spring includes a first conductive surface and a second conductive surface separate from the first conductive surface by non-conductive surfaces. The first cantilever spring includes a conductive trace with a plurality of conductive segments arranged on the conductive trace.

### CROSS-REFERENCE TO RELATED APPLICATION

This application claims the benefit of U.S. Provisional Application No. 63/407,003, filed Sep. 15, 2022, and titled “Upper Extremity Prosthetic Device,” which is incorporated by reference.

### FIELD

This specification generally relates to springs and prosthetics that include the springs.

### BACKGROUND

Three-dimensional (3D) printing is a widely used manufacturing technique for both commercial products and research across many industries with emerging research areas in multi-material 3D printing, metamaterials, and 3D printed electronics. Because of the high customizability and accessibility 3D printing offers, more mechanisms such as prosthetics are being 3D printed. However, conventional 3D printed prosthetics do not allow for the same function and control that a natural limb provides. For example, a natural limb allows a person to feel how hard they are grasping an object.

### SUMMARY

This specification generally describes springs that can be used to measure and/or detect physical properties and prosthetic devices that include the springs.

According to some implementations, an upper-extremity prosthetic includes a first member having a surface adapted to engage with an object; a first spring coupled to the first member, the first spring arranged to absorb energy and to provide energy return in response to movement of the first member, wherein the first spring has a first conductive surface and a second conductive surface separate from the first conductive surface by non-conductive surfaces; and a first cantilever spring extending from the first member and having a surface adapted to engage with the object while the object is also engaged with the surface of the first member.

Implementations may include one or more of the following features. In some aspects, the device includes a base member coupled to the first spring and pivotably coupled to the first member, where pivoting the first member relative to the base deflects the first spring. In some aspects, the device includes a prismatic joint element coupled to at least one end of the first spring. The prismatic joint element can be fully conductive.

In some aspects, the first spring is a coil spring. The coil spring can have a rectangular cross section. The coil spring can include a progressive pitch. In some aspects, the first cantilever spring includes a conductive material and a non-conductive material. The first cantilever spring can include a conductive base with multiple conductive segments arranged on the conductive trace. The first cantilever spring can include empty space between each pair of conductive segments.

In some aspects, the device includes a second spring and a second cantilever spring.

According to some implementations, a coil spring for measuring a physical property includes a coil having a first conductive surface, a second conductive surface, and one or more non-conductive surfaces that separate the first and second conductive surfaces.

According to some implementations, a cantilever spring for measuring a physical property includes a conductive material and a non-conductive material.

In some implementations, the prosthetic devices described herein can be made by a 3D printing (additive manufacturing) process. In particular embodiments, one or more of the springs of the prosthetic devices are made by a multi-material 3D printing process and include a first material that is electrically non-conductive and/or a second material that electrically conductive. In some embodiments, the prosthetic also includes one or more cantilevered springs that are also adapted to engage with the ball and to provide energy return while a user of the prosthetic is throwing the ball.

Particular embodiments of the subject matter described in this specification can be implemented to realize one or more of the following advantages. As described in this specification, some approaches for upper limb prosthetics fail to include energy store and return elements and result in limited functionality for the user of the prosthetic. Wearers (e.g., amputees) of upper limb prosthetics can perform tasks that demand substantial amounts of dexterity, fine control, and a number of degrees of freedom. The subject matter described in this specification provides wearers with an upper limb prosthetic that integrates energy storage and release elements to provide responsive, integrated sensing and control to the wearer of the upper limb prosthetic. Furthermore, the design of the upper limb prosthetic utilizes components that are more compact and customizable compared to existing prosthetics, e.g., through additive manufacturing techniques.

The application of the upper limb prosthetic described in this specification enables wearers of upper limb prosthetics to perform complex tasks and/or activities that demand wide ranges of motion and fine control. Wearers are enabled to perform complex tasks with improved range of motion and control through an energy return mechanism that enhances upper extremity prosthetics through the design of the described upper limb prosthetic. The energy return mechanism utilizes components such as springs, coils, cantilever springs, or some combination thereof, as a haptic feedback system to indicate to the wearer an amount of force applied by the prosthetic while performing a task. These components are made up of conductive materials to provide sensor signals to the wearer of the upper limb prosthetic, thereby providing embedded force sensing.

The use of one or more springs with integrated strain sensing capabilities for the prosthetic described herein may reduce the number of electronic components, reduce the steps for assembly after fabrication, and reduce the weight and overall cost in comparison to other techniques such as the use of a traditional accelerometer or pressure sensor.

The springs described in this specification can also be used for other applications, e.g., other than prosthetic devices. For example, the springs can be used to measure and detect various physical properties, such as weight, length changes and vibration. These measurement devices can be integrated in many different prosthetics and other applications where such physical properties are measured and/or detected.

The details of one or more implementations of the present disclosure are set forth in the accompanying drawings, and the description below. Other features and advantages of the present disclosure will be apparent from the description, drawings, and claims.

### BRIEF DESCRIPTION OF THE DRAWINGS

FIG. 1 depicts an example coil spring.

FIG. 2 depicts an example coil spring.

FIG. 3 depicts a graph of an example force-displacement evaluation for the coil spring of FIG. 2.

FIG. 4 depicts an example coil spring.

FIG. 5 depicts a graph of an example force-displacement resistance evaluation for the coil spring of FIG. 4.

FIG. 6 depicts an example coil spring.

FIG. 7 depicts a graph of an example force-displacement resistance evaluation for the coil spring of FIG. 6.

FIG. 8 depicts an example coil spring.

FIG. 9 depicts a graph of an example force-displacement resistance evaluation for the coil spring of FIG. 8.

FIG. 10 depicts an example coil spring.

FIG. 11 depicts an example coil spring.

FIG. 12 depicts a graph of an example force-displacement resistance evaluation for the coil spring of FIG. 11.

FIG. 13 depicts the mechanical structure of an example cantilever spring.

FIG. 14 depicts the cross sectional area of an example cantilever spring made with conductive material.

FIG. 15 depicts an example 3D printed cantilever spring.

FIG. 16 depicts a graph of an example force-displacement evaluation for the cantilever spring of FIG. 15.

FIG. 17 depicts an example 3D printed computational cantilever spring.

FIG. 18 depicts an example graph of force-displacement resistance evaluation for the computational cantilever spring of FIG. 17.

FIG. 19 depicts an example upper extremity prosthetic device.

FIG. 20 depicts movements of the upper extremity prosthetic device of FIG. 19.

FIG. 21 depicts an example coil spring.

FIG. 22 depicts a graph of an example force sensing signal for the coil spring of FIG. 21.

FIG. 23 depicts an example cantilever spring.

FIG. 24 depicts a graph of an example force sensing signal for the coil spring of FIG. 23.

FIG. 25 depicts an example system of signal communication between an upper extremity prosthetic device and a computing device.

FIG. 26 depicts an example display of sensor data from an upper extremity prosthetic device.

FIG. 27 depicts a graph of an example force sensing signal for an example coil spring and an example cantilever spring.

### DETAILED DESCRIPTION

An upper-extremity prosthetic device can be worn by a user (e.g., an amputee) to, for example, shoot a basketball into a basketball goal. The prosthetic device can include one or more springs, and can include a haptic feedback system that provides the wearer with a physical sensation that is proportional to the amount of force applied to the basketball via the prosthetic during the shot. Accordingly, the energy returned by the spring(s) may help the wearer shoot the basketball while using less energy, and the haptic feedback may help the wearer shoot the basketball with more fine-tuned control.

While the example scenarios described in this specification is a basketball shot, the design aspects of the prosthetic device described below can also be applied for prosthetics adapted for use in other contexts. For example, the concepts described herein can be applied in prosthetics adapted for purposes such as, but not limited to, throwing a football, throwing an item such as a baseball or softball, throwing a flying disc, dribbling a ball, and the like, without limitation.

This specification describes an energy return mechanism and a prosthetic hand that can be implemented by leveraging low-cost multi-material 3D printing to compensate for the forces and motion that would otherwise be generated by wrist and finger flexion. Embedded sensors measure the force generated through the prosthetic in real time. The embedded sensing allows designers to incorporate feedback, through haptics or other means, to an individual to finely adjust their motion. The energy return mechanisms have broad applicability for other prosthetic and non-prosthetic uses. The example techniques and designs described in this specification provide, for example, a method of combining multi-material 3D printing, 3D printed electronics and springs, compliant design and feedback to design energy return for upper limb prosthetics; and design guidelines and parameters to produce energy return elements with integrated sensing for feedback; characterization of the energy return and embedded sensing elements to create real time feedback within a prosthetic.

Coil springs and cantilever springs can be used to create functional energy return elements. This specification provides geometries (e.g., coil, cantilever), material (e.g., conductive, non-conductive) and sensing mechanisms (e.g., flex, surface contact) that enable effective implementation of these energy return elements in an upper limb prosthetic. The designs can achieve compactness, be 3D printed, have adjustable parameters and provide the necessary energy return and force sensing signal for effective use in upper extremity prosthetics.

Designing and fabricating a 3D printed spring can be a complex task due to varying material properties and printing constraints. This is compounded when using more than one material or if additional features, such as deflection sensing, are to be integrated.

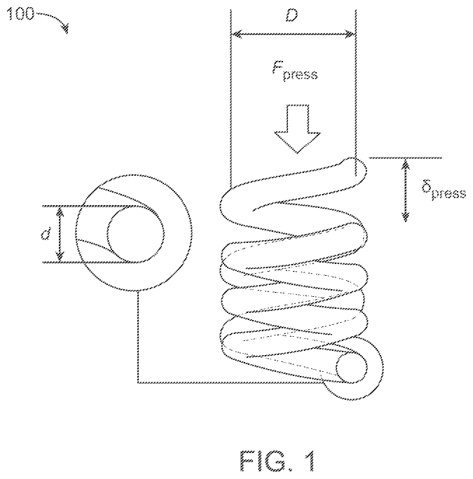

FIG. 1 depicts an exemplary mechanical structure of a coil spring 100, illustrating an application of Castigliano's theorem to describe the relationship between deflection of the coil spring 100 and any applied forces to the coil spring 100. The deflection of coil springs can be described by Castigliano's theorem below, relating the displacement δᵢ from a point i in which a force Fᵢ is applied to U, the total strain energy:

$${δ}_{i} = \frac{\partial U}{\partial {F}_{i}} \qquad ( 1 )$$

Additionally, the deflection-force relationship in a coil spring, e.g., coil spring 100, can also be represented by the following equation:

$${δ}_{press} = \frac{8 {F}_{press} {D}^{3} N}{{d}^{4} G} \qquad ( 2 )$$

The above equation relates the displacement δₚᵣₑₛₛ of the spring when force Fₚᵣₑₛₛ is applied, to the mean coil diameter D, the diameter of the coil cross section d, the number of active coils N, and the modulus of the coil's rigidity G—which can be based on material dependent properties such as Young's modulus and Poisson's ratio. Furthermore, other factors of the spring's design such as the mean coil diameter D, the number of active coils N, and the coil cross section diameter d can be adjusted to achieve a desired force Fₚᵣₑₛₛ and corresponding δₚᵣₑₛₛ for the spring.

In some implementations, the desired force Fₚᵣₑₛₛ and corresponding δₚᵣₑₛₛ are designed based on space constraints of the upper limb prosthetic. The energy stored in a coil spring (also referred to as the elastic potential energy P_{elastic}) can be described by a relationship between a spring constant k and deformation length Δx, of the spring:

P_{elastic}=½kΔx² (3)

The energy stored in a coil spring can be transformed into kinetic energy by designing coil springs, based on the intrinsic properties of the materials that make up the springs. Physical constraints such as size and form factor can be customized for applications, e.g., prosthetics, while the material properties of the spring enable delivery of target amounts of force or energy, e.g., to simulate different types of forces and motions experienced by complex joints.

FIG. 2 depicts an example coil spring 200. In this example, a base design for a coil spring is illustrated, in which the coil spring is not configured to perform embedded force sensing. The design of coil spring 200 includes a circular cross-section coil with a non-conductive spring 210 and a non-conductive prismatic joint 220, e.g., the coil spring is entirely made up of non-conductive PLA material. FIG. 3 depicts a graph 300 of example force calculations relative to displacement, for coil spring 200 of FIG. 2. The force calculations are based on the formulas (1)-(3) described above and empirical force measurements, e.g., by compressing coils to determine deflection. The improved stiffness and deflection illustrated by FIG. 3 can be attributed to printing resolution, e.g., sufficiently approximating the springs made up of natural materials such as metal, as well material property estimates of the coil spring, e.g., values utilized by formulas (1)-(3) may under-estimate intrinsic properties such as stiffness.

Example springs described in this specification can leverage the intrinsic properties of conductive polymer composites and printing techniques possible using fused deposition modeling (FDM). The conductive material detects stress and strain through a change in material resistivity. Multi-material printing with FDM allows for easy adjustments to printing parameters (e.g., infill, layer height, grid pattern) and concurrently print conductive and non-conductive structural materials. Along with designed dimensions, altering the infill percentage and material composition directly affects the mechanical structure, and thus energy storage, of a printed coil spring. The typical rough surfaces left by FDM printing also provide an approach to sense forces between conductive elements. As contact force is applied to a conductive PLA (cPLA) surface, the surface features flatten and the contact area increases, reducing electrical resistance. Both the intrinsic change in resistance and the surface roughness of printed cPLA can be used as sensing mechanisms for energy return coil springs.

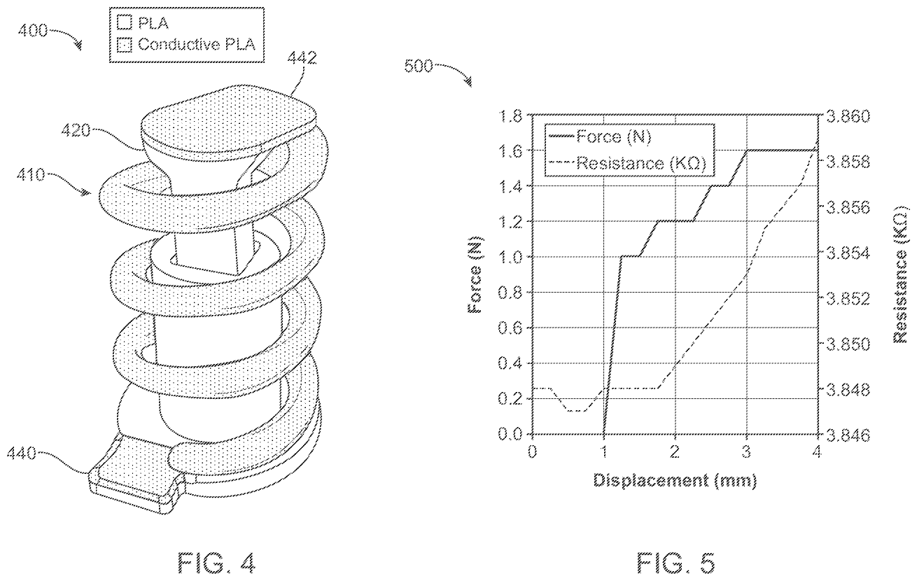

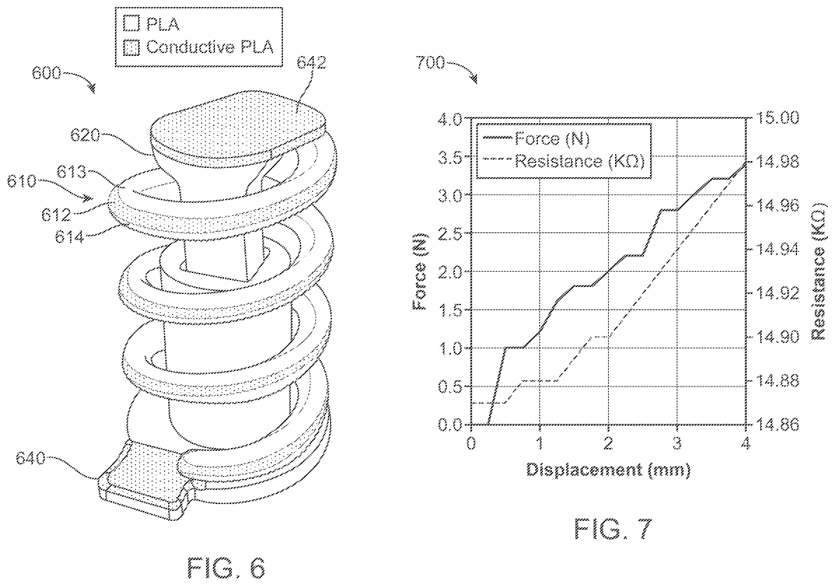

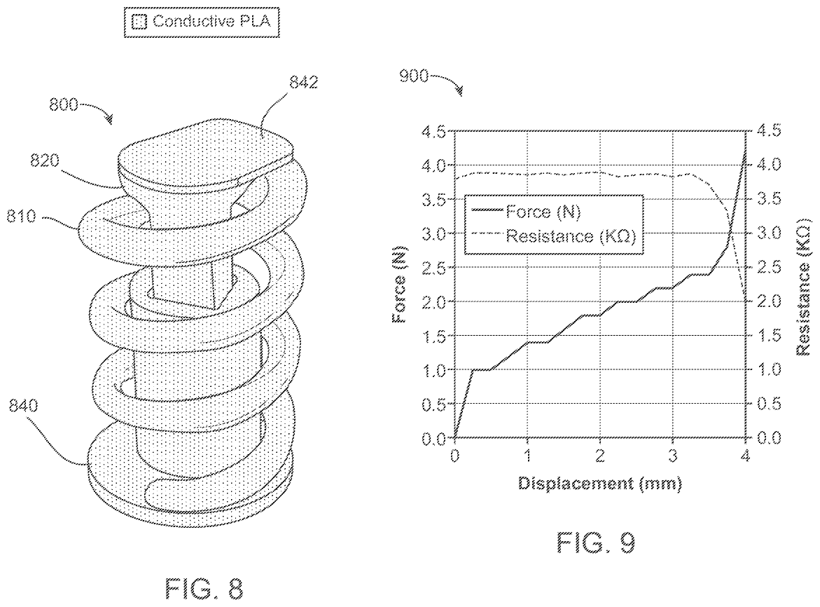

FIG. 4 depicts an example coil spring 400. In this example, cPLA can be added to the spring coil surface, creating a core/shell structure. This design includes a circular cross-sectional coil with a conductive spring 410 and non-conductive linear prismatic joint 420. Force sensing is provided through the resistance change of the coil conductor. The spring 410 also includes a first electrical contact 440 and a second electrical contact 442. The first electrical contact 440 and the second electrical contact 442 are each in electrical communication with the conductive material of the coil spring 410. The coil spring 400 can be an example design that demonstrates fewer interface issues between conductive and non-conductive portions of the coil spring 400, with a relatively low spring constant, e.g., compared to implementations described in reference to FIGS. 6, 8, etc. below.

FIG. 5 depicts a graph 500 of an example force-displacement evaluation for the coil spring 400 of FIG. 4. The graph 500 illustrates the relationship between force, displacement of the spring, and change in resistance from the applied force. The example evaluation depicted in the graph 500 illustrates a change in resistance that is approximately linear and proportional to the applied force for a conductive coil coupled to a non-conductive prismatic joint. The graph 500 also illustrates a relatively constant resistance (e.g., approximately 3.848 kΩ) at displacements approximately below 1.75 millimeters.

FIG. 6 depicts an example coil spring 600. This design includes partially conductive spring coil 610 and a non-conductive prismatic joint 620. In this example, the coils can be modified to a rectangular sandwich structure, rather than the circular structure of FIG. 4. The coils have at least one conductive area 612 and non-conductive areas 613 and 614. The spring 610 also includes a first electrical contact 640 and a second electrical contact 642. The first electrical contact 640 and the second electrical contact 642 are each in electrical communication with the conductive area 612 of the coil spring 610. Force sensing is done by monitoring the conductive coil's resistance. The coil spring 600 provides flexibility for energy storage, as the non-conductive area 613 and 614 of the spring coil can be tuned to provide desired mechanical properties and parameters, e.g., coil spacing, can match target displacement for devices.

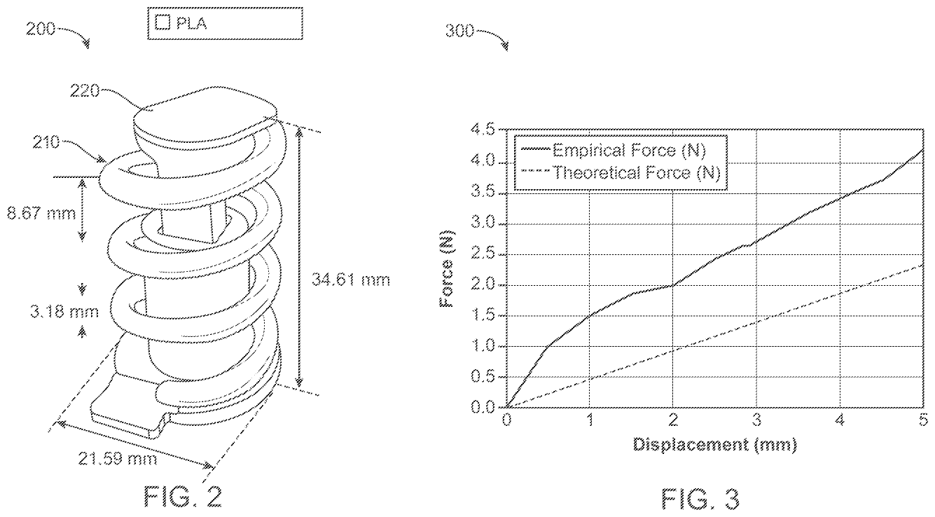

FIG. 7 depicts a graph of a force-displacement resistance evaluation for the coil spring 600 of FIG. 6. Similar to the graph 600, the example evaluation depicted in the graph 600 illustrates a change in resistance is approximately linear and proportional to the applied force. In contrast to graph 400, the graph 600 illustrates a resulting change in resistance across different values of displacement for the coil spring 600 having a partially conductive coil coupled to a non-conductive prismatic joint.

FIG. 8 depicts an example coil spring 800. This design includes a fully conductive coil 810 and a fully conductive prismatic joint 820. The spring 810 also includes a first electrical contact 840 and a second electrical contact 842. The first electrical contact 840 and the second electrical contact 842 are each in electrical communication with the conductive material of the coil spring 810. Force sensing is measured through resistance changes in both the coil spring 810 and the prismatic joint element 820. In some implementations, two print heads are used, such as a first print head for non-conductive PLA filament and a second print head for conductive composite (cPLA). The diameter of the nozzle for the print head can vary in size, e.g., based on a type of PLA or cPLA to be printed. For example, the composite cPLA can be printed using a standard 0.4 mm nozzle diameter. In some implementations, the coil spring 800 provides threshold force sensing—in which the conductive prismatic joint 820 is configured to provide feedback if an amount of force exceeding a threshold value of force is applied. Modifications to the prismatic structure of conductive prismatic joint 820 can provide, e.g., through force displacement of the attached spring coil 810, feedback signals that trigger at a range of input forces.

FIG. 9 depicts a graph 900 of a force-displacement resistance evaluation for the coil spring 800 of FIG. 8. In contrast to graphs 500 and 700, the example evaluation depicted in the graph 900 illustrates a sharp change in resistance at approximately 3.5 millimeters in displacement. The graph 900 illustrates a displacement that is approximately linear and proportional to the applied force. The graph 900 illustrates a resulting change in resistance across different values of displacement for the coil spring 800 having a conductive coil coupled to a conductive prismatic joint.

Modifying the material makeup and geometry of the springs modifies the mechanical properties, e.g., as illustrated in FIGS. 4, 6, and 8 for corresponding coil springs 400, 600, and 800, respectively. The coil springs 400, 600, 800 demonstrate the force-displacement-resistance changes respectively illustrated in the graphs 500, 700, and 900, which can be provided for force sensing in a device, e.g., an upper limb prosthetic.

The changes in the force sensing signal illustrated in both FIG. 5 and FIG. 7 are linear and 1% of the total resistance at maximum deflection of the respective coil spring. The springs provide a resistance change that can be mapped to input force for detection. The sharp change in resistance seen in FIG. 9, occurs precisely when the trapezoidal geometry at the top of the spring makes contact with the top sleeve of the prismatic joint 820. In some implementations, the type of design illustrated in FIG. 8 for coil spring 800 can be useful for systems that require a threshold or binary output in force detection.

FIG. 10 depicts an example coil spring 1000. The coil spring 1000 is a constant pitch spring 1010 with a rectilinear cross-section but with the inverted sandwich of FIG. 6, creating two conductive surfaces 1013 and 1014 that spiral down the coil 1010 as shown in FIG. 10. The conductive surfaces 1013 and 1014 are separated by a non-conductive surface 1012 or area between the two conductive surfaces 1013 and 1014. Resistance is measured between the top and bottom conductive path, which operates as an open circuit when no force is applied. As the spring is compressed, the two conductors come into contact and a change in resistance can be observed. In some implementations, this configuration provides a sharp change in resistance near its maximum compression, when all coils make contact simultaneously, bridging the top and bottom conductive paths along the full length of the coil.

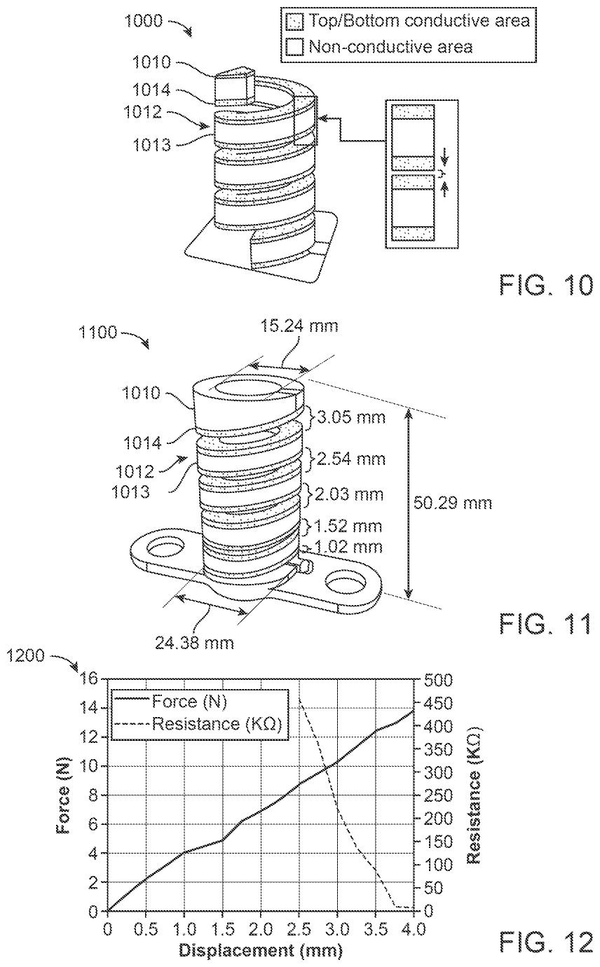

To create a more continuous signal change, the coil spring 1000 of FIG. 10 can be modified to include a progressive pitch, as shown in the coil spring 1100 of FIG. 11. With a progressive pitch, the coils make electrical contact ordinally (e.g., in an ordinal direction) from bottom to top, providing a continuously changing signal. This spring 1100 has two observable output states as force is applied. Initially, when the spring 1100 compresses and creates a connection between the upper and lower electrodes, there is a rapid decrease in measured resistance. As the distance between all coils compresses to zero, the rough deformable surfaces of the spring continue to compress, further reducing the resistance but at a significantly smaller slope when resistance is plotted against force, as shown in FIG. 12. In some implementations, the coil spring 1100 demonstrates resistance changes that can be mapped to applied force, e.g., the progressive pitch of the coil spring 1100 demonstrating a higher resolution signal and additional information on the spring state.

The spring 1100 also introduces substantial hysteresis that can be seen during rapid press and release of the spring. This hysteresis also reduces the forces produced, as coils stick together and reduce returned power. A change in resistance can be read with respect to force through the full compression cycle of the spring. From an initial open circuit, the spring 1100 starts at a resistance of about 35 kΩ when lightly compressed and the bottom two coils begin making contact with one another. When fully compressed, the spring 1100 has a minimum resistance of about 3.6 kΩ. In some implementations, the spring 1100 can provide a higher spring constant relative to designs for coil springs illustrated in the previous figures, e.g., FIG. 4, 6, or 8.

FIG. 12 depicts a graph 1200 of an example force-displacement resistance evaluation for the coil spring of FIG. 11. In contrast to graphs 500, 700, and 900, the example evaluation depicted in the graph 1200 illustrates an open circuit for displacements below 2.5 millimeters. As contact is made at the lower coil, the resistance of the contacts decreases linearly. For example, the graph 1200 illustrates a resistance of 457 kΩ upon contact between the coils when the coil spring 1100 is initially compressed. As the spring initially compresses and creates a connection between upper and lower electrodes, e.g., conductive surfaces 1013 and 1014, the resistance rapidly decreases. At full compression of the coil spring 1100, the graph 900 illustrates a resistance of 7.3 kΩ. As the distance between all coils compresses to zero, the rough deformable surfaces of the spring 1100 continue to compress, thereby further reducing the resistance at a smaller rate of change, e.g., compared to light compression of the spring 1100 slope.

Each configuration of coil spring described above provides a potential energy return element for upper limb prosthetics and other applications. The coil spring 800 of FIG. 8, which incorporates a conductive prismatic joint can be designed to provide threshold force sensing, providing an alert if an amount of force that exceeds a threshold value of force is applied. By modifying the prismatic structure, coupled to the force displacement of the attached spring, the output signal can be tuned to trigger at a range of input forces. The surface contact springs of FIGS. 10 and 11 also provide the resistance changes that can be mapped to applied force, with the progressive spring demonstrating a higher resolution signal and additional information on the spring state, such as “in compression” versus “fully compressed” based on the force-resistance relationship. Both the fully conductive and the sandwiched conductive springs 600 and 800 of FIGS. 6 and 8, respectively, provide proportional output with force. The coil spring 800 of FIG. 8 can provide a simpler design with fewer issues with material interface, but the coil spring 600 of FIG. 6 can provide more flexibility in energy storage as the non-conductive components of the spring coil can be tuned to provide the necessary mechanical properties, e.g., stiffness, durability, coil-spacing, and amount of energy return.

Fabrication-related parameters such as infill pattern, infill density, printing direction, and printing layering height, can also affect mechanical properties of the spring coil. In some implementations, printers with two or more printer heads can be utilized for fabricating springs, e.g., to add filament for additional elasticity in the spring. In some implementations, springs can be fabricated with filaments derived from materials with a lower flexural modulus than PLA, such as thermoplastic polyurethane (TPU). For example, flexible filaments made from TPU can provide additional flexibility to portions of the spring made from TPU compared to other portions of the spring made from PLA or cPLA. The additional flexibility, e.g., from additional elasticity in the springs, through flexible filaments made up of TPU can provide more friction, e.g., improving grip, in portions of the spring in which the flexible filaments are used. Furthermore, dissolvable support materials such as polyvinyl alcohol (PVA) can be used as support material to print complex geometries in fabricating the spring.

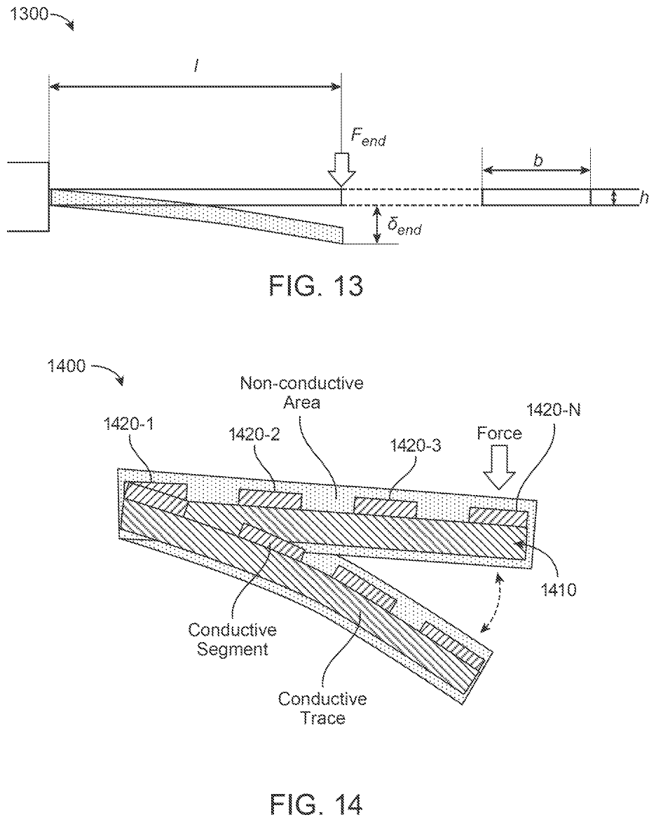

FIG. 13 depicts the mechanical structure of an example cantilever spring 1300. FIG. 14 depicts the cross-sectional area of an example cantilever spring 1400 made with conductive material. The example cantilever spring 1400 includes a number “N” of conductive segments 1420-1 through 1420-N arranged on a conductive base 1410. Non-conductive areas can be used to separate the conductive segments 1420. Mechanical properties can be controlled by adjusting the cantilever dimensions, while considering the energy loss in these structures. The deflection of the cantilever spring 1300 can be described by Castigliano's theorem, in which the maximum deflection at the end of the cantilever δ_{end} is determined by the length/of the cantilever, Young's modulus E, second area moment I of the cantilever cross-section, and the force at the end of the cantilever F_{end}:

$${δ}_{end} = \frac{{F}_{end} {l}^{3}}{3 EI}$$

The second area moment of the cantilever cross-section is based on the width b and the height h of the cantilever spring:

$$I = \frac{{bh}^{3}}{12}$$

Additionally, the strain energy absorbed by the cantilever spring 1300 can be described by the following equation:

$$U = \frac{{F}_{end}^{2} {l}^{3}}{6 EI}$$

In some implementations, the cantilever spring 1300 is configured to provide a portion of stored energy for a device, e.g., prosthetic device. For example, the cantilever spring 1300 can be configured to mimic lower force joints, e.g., such as fingers, in a prosthetic device. Furthermore, the cantilever spring 1300 can be embedded within existing structures to provide routing for energy return for a prosthetic, without drastic design and size changes for the prosthetic due to the simplicity and versatility of the cantilever spring.

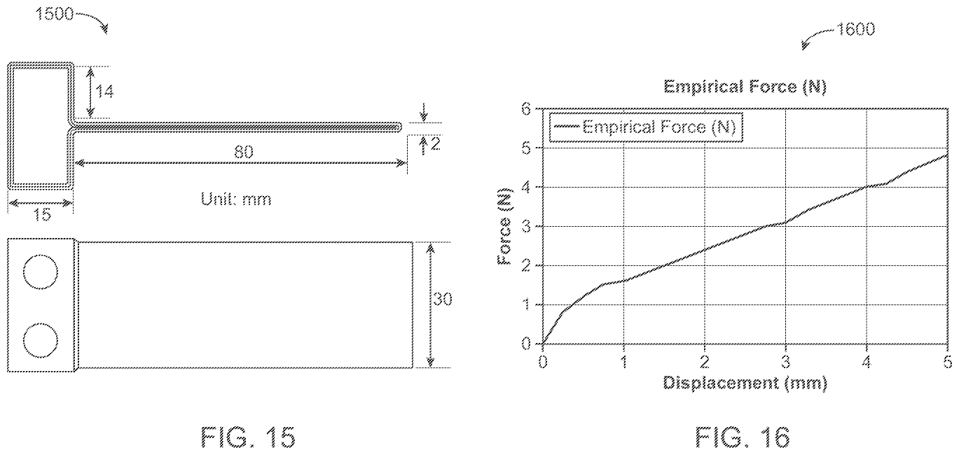

FIG. 15 depicts an example 3D printed cantilever spring 1500 and example dimensions for the cantilever spring 1500. This cantilever spring 1500 can be used as a base for a computation cantilever spring described below. The cantilever spring 1500 illustrates a smaller form factor and more simple design compared to the coil springs, e.g., coil spring 800, described above. Dimensions for cantilever spring 1500 are illustrated as an example, although any size of cantilever can be used based on the target mechanical properties, e.g., considering energy loss as the size of the cantilever increases. Dimensions for the cantilever spring can also be based on a type of application, such as utilizing large springs for industrial machines compared to smaller springs, e.g., for wearable prosthetic devices.

FIG. 16 depicts a graph 1600 of an example force-displacement evaluation for the cantilever spring 1500 of FIG. 15. This graph 1600 displays the relationship between force and displacement, illustrating an example demonstration of Castigliano's Theorem, e.g., a deflection-force relationship by relating the displacement from which a force is applied in the spring and the total strain energy of the spring.

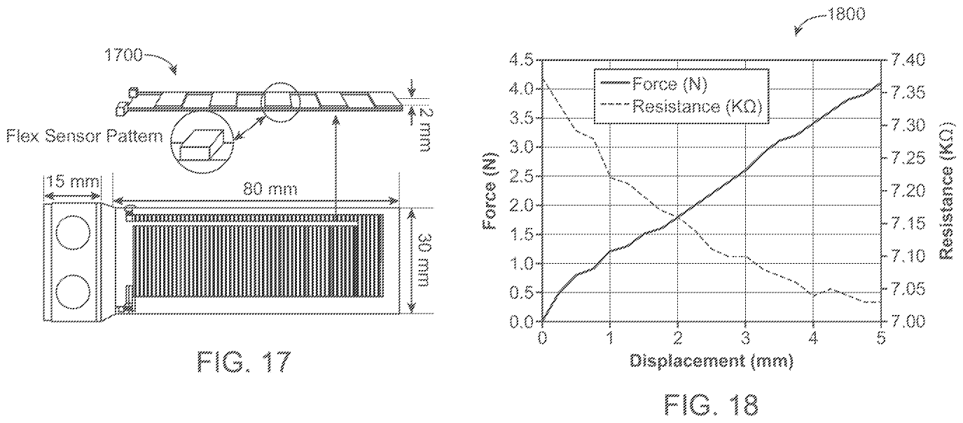

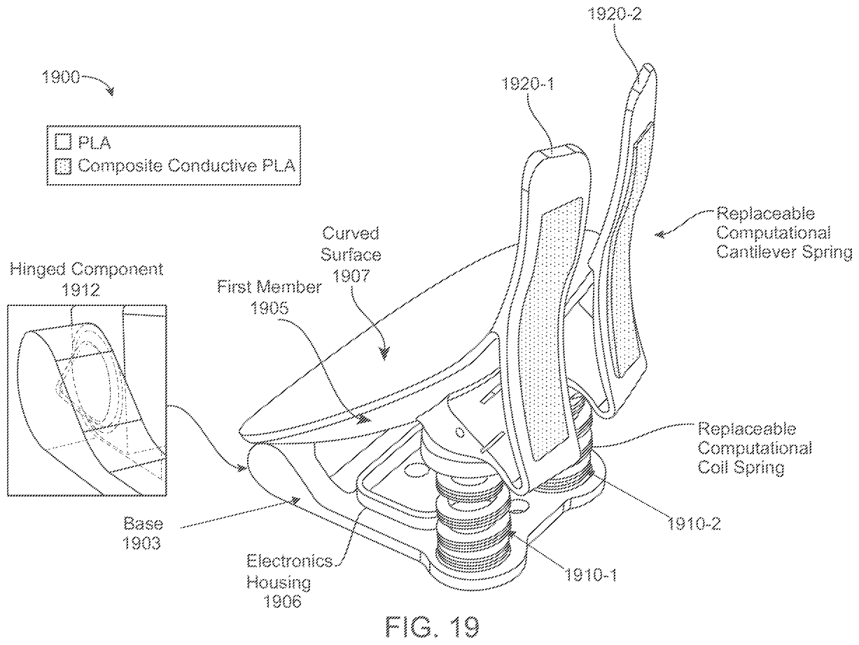

FIG. 17 depicts an example 3D printed computational cantilever spring 1700 and example dimensions for the computational cantilever spring 1700. In this example, conductive elements 1710 are integrated with the base cantilever spring 1700. Fundamentally, the resistance value of a flex sensor varies according to its bending angle, and the cantilever spring's bend is determined by the applied force. By combining these unique characteristics, a force-sensitive cantilever spring 1700 can be created. FIG. 18 depicts an example graph 1800 of force-displacement resistance evaluation for the computational cantilever spring 1700 of FIG. 17. This graph 1800 displays the relationship between force, displacement of the spring, and resulting changes in resistance. For example, the graph 1800 illustrates that the resistance decreases as deflection of the cantilever spring 1700 increases.

The cantilever spring 1700 can include conductive and non-conductive materials. For example, the areas between conductive elements can include non-conductive materials. The computational cantilever spring 1700 can be used to measure various physical properties, such as weight, length change, vibration, etc. The design of the cantilever spring 1700 enables size adjustments that enable modifications to spring constant, energy storage, and other related mechanical properties. Integrating cPLA, e.g., conductive materials, to configure the cantilever spring 1700 for force-sensing demonstrates, e.g., by the graph 1800, provides that the force and displacement can be detected through changes in the conductive materials. In some implementations, energy storage and force sensing for the cantilever spring 1700 can be tuned to meet target mechanical and electrical specifications. For example, mechanical and electrical specifications can include peak energy storage, variable resistance range, average resistance, stiffness and other functional properties.

FIG. 19 depicts an example upper extremity prosthetic device 1900. The upper extremity prosthetic device 1900 includes a base 1904 with an electronic housing 1906, a first member 1905, two coil springs 1910-1 and 1910-2, and two cantilever springs 1920-1 and 1920-2. The coil springs 1910-1 and 1910-2 can be implemented using any of the coil springs described in this document. Similarly, the cantilever springs 1920-1 and 1920-2 can be implemented using any of the cantilever springs described in this document. A hinged component 1912 is illustrated in FIG. 19, connecting the first member 1905 to the base 1903 that includes the two coil springs 1910-1 and 1910-2. The electronic housing 1906 can include a haptic device that provides haptic feedback to a wearer of the upper extremity prosthetic device 1900 based on force sensing signals received from the springs 1910 and/or 1920. In some implementations, the electronic housing can include an electrical sensing systems that receives signals from cantilever springs 1920 and coil springs 1910.

The first member 1905 has a surface 1907 adapted to engage with an object, e.g., an athletic ball. The coil springs 1910 can be coupled to the first member 1905 and the base 1903 and can include a prismatic joint element. Each coil spring 1910 can be configured to provide energy return in response to movement of the first member 1905. Each coil spring 1910 can have a first conductive surface and a second conductive surface separate from the first conductive surface by non-conductive surfaces. Each cantilever spring 1920 can extend from the first member 1905 and have a surface adapted to engage with the object while the object is also engaged with the surface 1907 of the first member 1905.

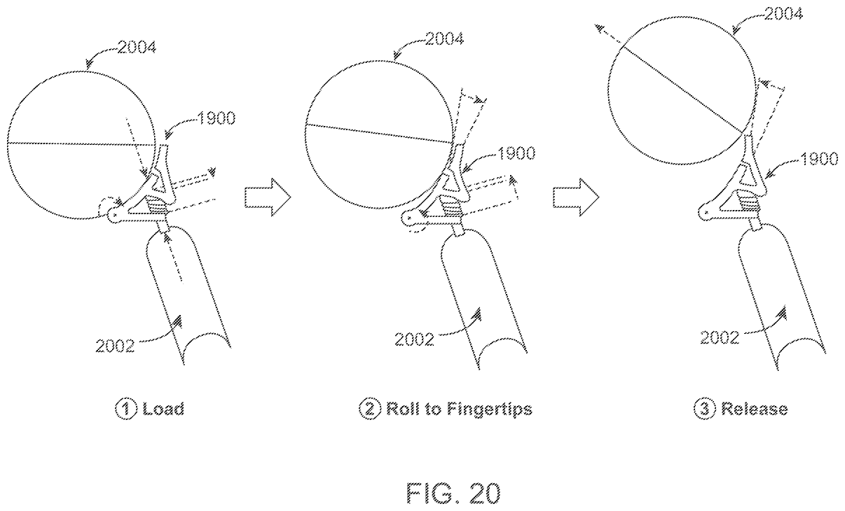

FIG. 20 depicts movements of the upper extremity prosthetic device 1900 of FIG. 19.

In this example, the coil springs 1910 provide compensation for expected wrist motion during a shot, while the cantilever springs 1920 can be positioned to compensate for fingers as the ball leaves the hand. Multiple variables contributing to a successful basketball shot can include the amount of force provided, angle of approach, etc. Some variables that significantly impact the success of a basketball shot are the release height, release velocity and angle of release. Lower limbs are primarily responsible for shooting distance, while upper limbs provide fine tuning of shooting movement. Shot accuracy is determined by a kinematic chain that includes multiple degrees of freedom: shoulder, elbow, wrist and finger flexion, which aim and release the ball to create the proper angle and introduce rotation in the shot.

Immediately before the forward motion of the shot begins, the shooting arm 2002 is in a loaded position. This position has the shoulder above 90°, the elbow in flexion, the wrist in almost full extension, and the fingers in close to full extension but in contact with the ball. To generate the forward and upward velocity required to aim and shoot the basketball 2004, elbow extension occurs first, increasing the forces applied by the hand on the ball, with wrist flexion shortly after, providing vertical and horizontal forces respectively. As the elbow continues to extend and the wrist flexes, the ball rolls closer to the fingertips causing a small amount of finger extension. Finger flexion applies the last bit of force to the ball as it leaves the shooter's hand, generating backspin for accuracy. The index and middle or middle and ring fingertips are the last two points to make contact with the basketball 2004. By applying different amounts of force with each finger, the player can make very small corrections that affect the trajectory of the ball.

The upper extremity prosthetic device 1900 of FIG. 19, which can be attached to a prosthetic wrist, mimics these biomechanics. As mentioned, the force applied to the ball by the hand from wrist flexion creates motion towards the basket; thus, the force is roughly parallel to the horizontal plane when released while fingers introduce spin and provide the last point of contact prior to release. Both wrist and finger flexion are active movements where muscles contract to generate the necessary motion and apply force to the basketball 2004. The upper extremity prosthetic device 1900 allows an individual with transradial limb loss to more naturally shoot a basketball 2004.

The upper extremity prosthetic device 1900 includes two energy return coil springs 1910-1 and 1910-2 to compensate for wrist flexion and two cantilever springs 1920-1 and 1920-2 to mimic fingers. The cantilever springs 1920 are offset from the main load path to delay the force applied by finger flexion, replicating a natural shot. The coil springs 1910 are compressed and the shooter moves upward, generating force primarily from their legs. As the wearer pushes the ball upward, and the coil springs 1910 release, the ball rolls up to the fingers forcing the cantilever springs 1920 to bend backwards. When the wearer's arm 2002 begins to slow, the cantilever springs 1920 apply force to the ball creating backspin.

As the index and middle, or middle and ring, fingers are the last two points to make contact with the basketball 2004, the upper extremity prosthetic device 1900 can include two fingers with two pairs of springs 1910 and 1920 each. Focusing on the hand and wrist, a basketball shot consists of three phases: load, roll to fingertips, and release. In the load phase of the shot, as the elbow begins to extend, the wrist is in near full extension. In the design of the upper extremity prosthetic device 1900, the coil springs 1910 are compressed, causing the ball to sink deeper into the prosthetic just like it would sink deeper into the palm of the shooter's hand. As the wrist flexes, the ball rolls up to the shooter's fingers as a small amount of finer extension occurs.

As the shooter's arm 2002 begins to decelerate, the coil springs 1910 release causing the ball to roll up to the prosthetic fingers, which extend. Two fingertips provide the last points of contact with the ball. The finger flexion exerted by these figures on the ball is what generates backspin. The upper extremity prosthetic device 1900 functions the same way with the cantilever springs 1920 releasing, redirecting the force on the ball to generate backspin. The cantilever springs 1920 are designed to match the size and placement of a shooter's hand on the ball.

The upper extremity prosthetic device 1900 mimics the natural motion of a basketball shot but can be printed in five parts: the main body, two computational coil springs 1910, and two cantilever springs 1920. In some implementations, the upper extremity prosthetic device 1900 can be printed in a single part, e.g., by adding support material to print the parts together. In some implementations, a main body includes the first member 1905, base 1904, and electronics housing 1906. In some implementations, the main body includes the hinged component 1912, e.g., an integrated printed conical joint, that the prosthetic rotates about, similar to a human wrist. The main body contains an integrated space to house an electrical sensing system that receives signals from each energy return element (e.g., each spring). As an example, the entire upper extremity prosthetic device 1900, including electronics, can weigh about 7 oz, making it lightweight for use.

In some implementations, the weakest point of a 3D-printed model is between layer lines along the z-axis. Therefore the cantilever springs 1920 can be printed independently from the rest of the upper extremity prosthetic device 1900. Doing so allowed for them to be printed horizontally, meaning each printed layer could run the length of the finger for increased durability and energy return. The fingers can each be attached to the body by using any type of fastening mechanism such as screws, nuts, or some combination thereof. The upper extremity prosthetic device 1900 can introduce force in line with a typical shot at 45 degrees as opposed to the roughly forward force generated by a wrist during a shot.

A wearer of the upper extremity prosthetic device 1900 can receive feedback data (e.g., plots, figures, instructions) on the amount of force being applied to the ball through their palm and fingers. Feedback data can enable the wearer to make fine adjustments to the ball mid-shot, such as applying more force through one finger over another, to increase accuracy. For example, a wearer can receive feedback data related to an orientation of the ball, e.g., basketball 2004, and the force being applied through the first member and cantilever springs of the upper extremity prosthetic device. Applied forces can be mapped to generate haptic or other feedback during use.

Feedback can be customized based on multiple types of signals corresponding to different types of springs to best inform a wearer while a shot is being performed. As an example, overall balance of a shot can be reported by taking a constant differential of the bilateral coil springs. In a haptic feedback implementation, vibration can indicate forces by positioning motors on either side of the arm 2002. In some implementations, feedback can be customized for each spring in the upper extremity prosthetic device 1900. Furthermore, force sensor signals can be mapped to outputs, e.g., streams of feedback data provided to a device that the wearer can review, thereby providing the wearer a model of feedback preferences, e.g., an amount of feedback provided for the most accurate shot.

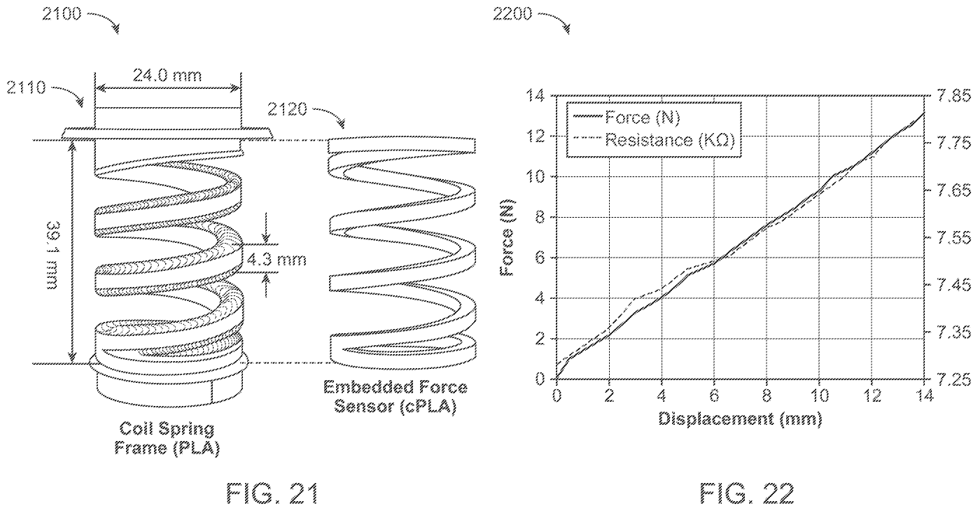

FIG. 21 depicts an example coil spring 2100 based on a sandwich structure design, e.g., similar to FIG. 6. The coil spring 2100 includes non-conductive material 2110 and an embedded force sensor 2120 made up of conductive material, e.g., cPLA. The conductivity of the embedded force sensor 2120 provides that an amount of force experienced by the coil spring 2100 translates to an amount of resistance corresponding to the amount of force. Measuring changes in resistance can provide force sensing, e.g., detecting changes in the amounts of force.

FIG. 22 depicts a graph 2200 of a force sensing signal for the coil spring 2100 of FIG. 21. The graph 2200 illustrates a resulting change in resistance across different values of displacement for the coil spring 2100. For example, graph 2200 illustrates a linear relationship between displacement and force, correlating to the amount of resistance provided through force sensing components of the spring 2100, e.g., embedded force sensor 2110. Furthermore, graph 2200 illustrates an increased spring constant and achieves maximum displacement for the coil spring 2100, e.g., achieving full compression and decompression.

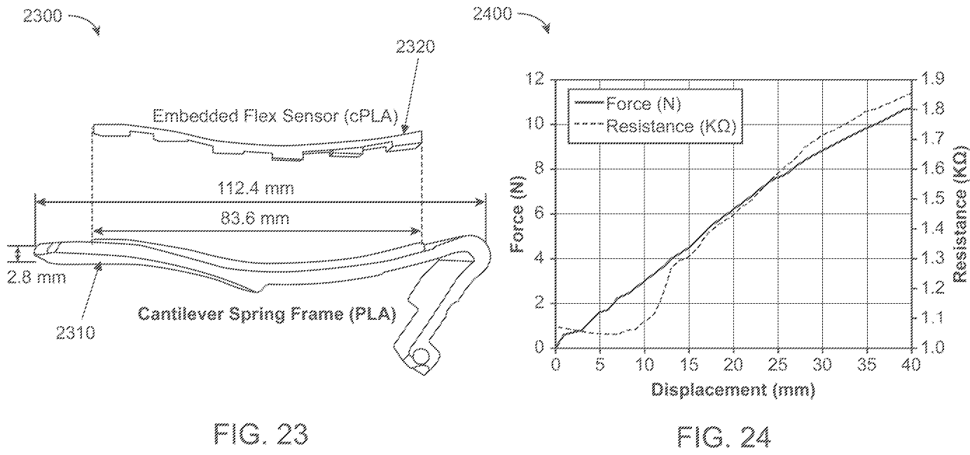

FIG. 23 depicts an example cantilever spring 2300 that includes non-conductive material 2310 and an embedded flexible force sensor 2320 made up of conductive material, e.g., cPLA. Similar to FIG. 21, the embedded flexible force sensor 2320 provides that changes in resistance indicate changes in the amounts of force experienced by the cantilever spring 2300.

FIG. 24 depicts a graph 2400 of a force sensing signal for the cantilever spring 2300 of FIG. 23. The graph 2300 illustrates a resulting change in resistance across different values of displacement for the cantilever spring 2300. For example, graph 2400 illustrates a linear relationship between displacement and force, correlating to the amount of resistance provided through force sensing components of the spring 2300, e.g., embedded flexible force sensor 2320. Furthermore, graph 2400 illustrates an increased spring constant and achieves maximum displacement for the cantilever spring 2300, e.g., achieving full compression and decompression.

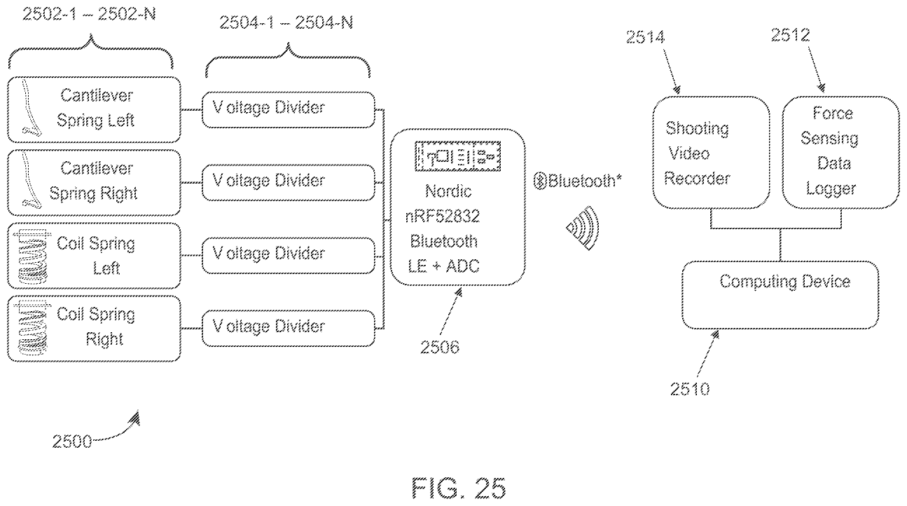

FIG. 25 depicts an example system 2500 configured to communicate signals between an upper extremity prosthetic device 1900 and a computing device 2510. The upper extremity prosthetic device 1900 can include any number of springs 2502-1 through 2502-N, e.g., coil springs, cantilever springs, although two cantilever springs and two coil springs are illustrated. The system 2500 includes a corresponding voltage divider 2504 for each spring, e.g., voltage dividers 2504-1 through 2504-N. The voltage divider 2504 for a spring provides a conduit to obtain a signal from a respective spring and transmits the respective signal. For example, the voltage divider 2504 is stored in the electronics housing 1906 for the upper extremity prosthetic device 1900. Upon obtaining a signal from a corresponding sensor, e.g., force-based sensing through conductive portions of a spring, the signal can be transmitted by a communication device 2506. The communication device 2506 can include any appropriate form of wireless transmission, e.g., Bluetooth, to establish a connection between the upper extremity prosthetic device 1900 and the computing device 2510.

Signals from the upper extremity prosthetic device 1900 to the computing device 2510 can be processed by a force sensor data logger 2512, e.g., coupled to the computing device 2510 or part of the computing device 2510. The computing device 2510 can also include a camera device 2514 to capture footage of a wearer of the upper extremity prosthetic device 1900. Footage that includes one or more images captured by the camera device 2514 can be coupled to measurement data captured by the force sensing data logger 2512. By coupling image and/or video data to force sensor measurements, a wearer can simultaneously review visual data and measurement data to adjust/tune the springs of the upper extremity prosthetic device 1900. In some implementations, the computing device 2510 is a mobile device, e.g., a smartphone, a tablet. The computing device 2510 may be any device capable of wirelessly receiving signals. In some implementations, the camera device 2514 is a separate device from the computing device 2510.

The system 2500 can be configured as a real-time monitoring system of energy return for the upper extremity prosthetic device 1900. In some implementations, the voltage dividers 2504-1-2504-N include analog-to-digital converters to digitize samples of sensor measurements from springs of the upper extremity prosthetic device 1900. In some implementations, the communication device 2506 includes a microcontroller device with one or more cores. The microcontroller device can include a core for collecting converted, e.g., digitized, samples and generating packets of data that include the digitized samples. Additional cores can be designated for transmitting the packets of data to a connected device, e.g., computing device 2510.

In some implementations, sample measurements of the coil-based sensors of the upper extremity prosthetic device 1900 are collected as calibration data to configure the upper extremity prosthetic device 1900 to the wearer's preferences. For example, preferences can include responsiveness and other types of energy return parameters. Changes in force/resistance from the upper extremity prosthetic device 1900 can be measured by sensors in real-time, and coupled with simultaneously captured footage.

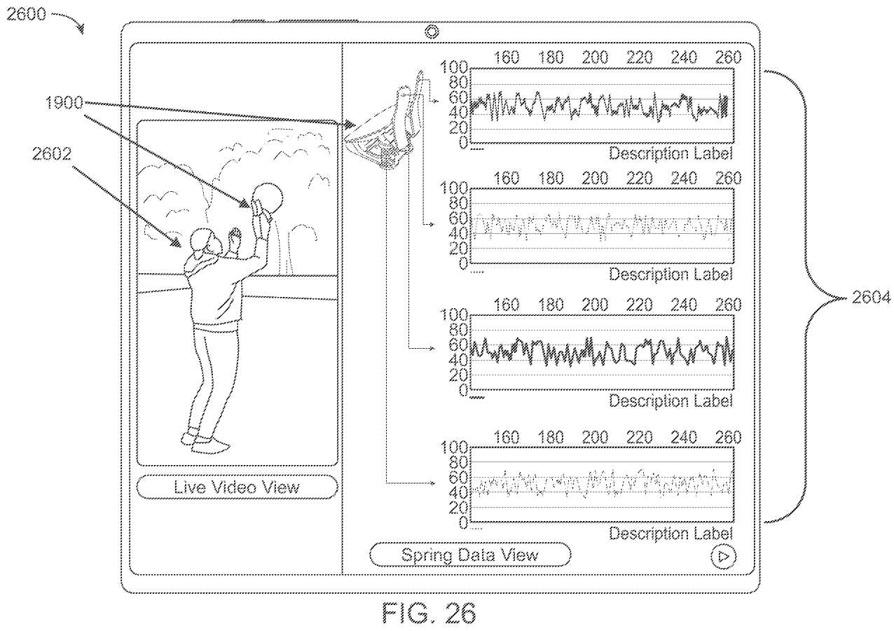

FIG. 26 depicts an example display 2600 of sensor data from an upper extremity prosthetic device. The display 2600 illustrates footage 2602 of a wearer of the upper extremity prosthetic device 1900 with the graphs 2604 illustrating sensor data from different springs that are included in the upper extremity prosthetic device 1900.

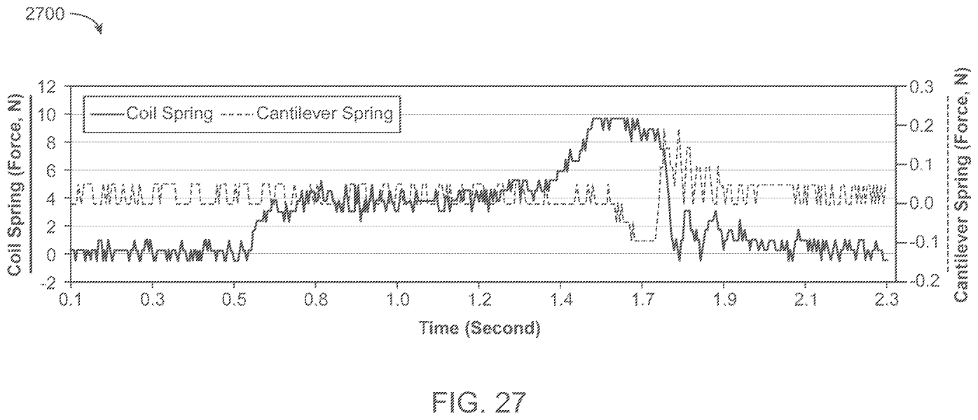

FIG. 27 depicts a graph 2700 of an example force sensing signals from coil springs and cantilever springs of the upper extremity prosthetic device 1900. As illustrated by the graph 2700, data from the sensors of the upper extremity prosthetic device 1900 can be categorized by type of spring, e.g., cantilever, coil, and be associated to a particular spring. For example, the graph 2700 illustrates a signal from one of the two coil springs and another signal from one of the two cantilever springs, of the upper extremity prosthetic device 1900. The graph 2700 illustrates force applied onto the springs during one or more actions performed by the wearer using the upper extremity prosthetic device 1900, e.g., carrying an object, throwing an object. The graph 2700 may be part of an example display, e.g., display 2600, on a computing device, screen, monitor, etc.

While the spring designs described herein are applied to an upper limb prosthetic device, the design principles are applicable to general prosthetic applications and beyond. Sports such as baseball, bowling, and frisbee all require fine control of wrist and finger motions. In these applications, custom energy return designs can be implemented to generate the appropriate amount of force during play while providing force feedback signals for fine tuning or haptics integration. Outside of sports, there are other applications where an energy return system such as those described in this specification could be used in upper limb prosthetics. Actions such as precision grasping often move very slowly. Adding an energy return component may improve performance and make various daily life activities easier to achieve. Some examples include being able to push open a heavy door more efficiently, quickly generating extra grip strength to turn an otherwise stuck lid or pressing a button at a challenging angle. By understanding the force dynamics in the energy return components, springs can be designed to store and release energy when needed.

Using energy return shoes as an example, pressure distributions can be recorded from the runner's stride through the springs themselves without the need of an external sensor by instrumenting energy return elements into the midsoles with a conductive polymer composite material. Using this information, the timing of the energy release can be optimized for greater energy return and may lead to more accurate step counts and activity classification. Additionally, real-time sensing could be provided to the wearer, which may help improve a wearer's stride and avoid injury.

Each spring described in this specification can be 3D printed using, for example, a multi-material 3D printing (additive manufacturing) process.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. An upper-extremity prosthetic comprising:
a first member having a surface adapted to engage with an object;
a first coil spring coupled to the first member, the first coil spring arranged to absorb energy and to provide energy return in response to movement of the first member, wherein the first coil spring comprises a first conductive surface and a second conductive surface separate from the first conductive surface by non-conductive surfaces; and
a first cantilever spring extending from the first member and having a surface adapted to engage with the object while the object is also engaged with the surface of the first member, wherein the first cantilever spring comprises a conductive trace with a plurality of conductive segments arranged on the conductive trace.

2. The upper-extremity prosthetic of claim 1, further comprising a base member coupled to the first coil spring and pivotably coupled to the first member, wherein pivoting the first member relative to the base member deflects the first coil spring.

3. The upper-extremity prosthetic of claim 1, further comprising a prismatic joint element coupled to at least one end of the first coil spring.

4. The upper-extremity prosthetic of claim 3, wherein the prismatic joint element is fully conductive.

5. The upper-extremity prosthetic of claim 1, wherein the first coil spring has a rectangular cross section.

6. The upper-extremity prosthetic of claim 1, wherein the first coil spring comprises a progressive pitch.

7. The upper-extremity prosthetic of claim 1, wherein the first cantilever spring comprises a conductive material and a non-conductive material.

8. The upper-extremity prosthetic of claim 7, wherein the conductive material is conductive polylactic acid and wherein the non-conductive material is polylactic acid.

9. The upper-extremity prosthetic of claim 1, wherein the first cantilever spring comprises a plurality of openings, each opening in the plurality of openings disposed between each pair of conductive segments.

10. The upper-extremity prosthetic of claim 1, further comprising a second coil spring coupled to the first member and a second cantilever spring extending from the first member and having a surface adapted to engage with the object while the object is also engaged with the surface of the first member.

11. The upper-extremity prosthetic of claim 1, further comprising at least one processor coupled to at least one memory storing instructions, wherein the at least one processor, upon executing the instructions, performs operations comprising:
transmitting one or more signals based on measured resistance from at least one of (i) the first coil spring, or (ii) the first cantilever spring;
analyzing the one or more signals; and
generating haptic feedback based on the one or more signals.

12. The upper-extremity prosthetic of claim 10, further comprising at least one processor coupled to at least one memory storing instructions, wherein the at least one processor, upon executing the instructions, performs operations comprising:
transmitting one or more signals based on measured resistance from at least one of (i) the first coil spring, (ii) the first cantilever spring, (iii) the second coil spring, or (iv) the second cantilever spring;
analyzing the one or more signals; and
generating haptic feedback based on the one or more signals.

13. A coil spring for measuring a physical property, the coil spring comprising:
a coil having a first conductive surface, a second conductive surface, and one or more non-conductive surfaces that separate the first and second conductive surfaces.

14. The coil spring of claim 13, wherein the coil of the coil spring has a rectangular cross section.

15. The coil spring of claim 13, wherein the coil of the coil spring comprises a progressive pitch.

16. The coil spring of claim 13, wherein at least one or more of the first conductive surface or the second conductive surface comprises conductive polylactic acid, and at least one of the one or more non-conductive surfaces comprises polylactic acid.

17. The coil spring of claim 13, wherein the coil spring is configured to generate an electrical signal in response to a force applied to the coil of the coil spring.

18. A cantilever spring for measuring a physical property, the cantilever spring comprising:
a conductive trace with a plurality of conductive segments arranged on the conductive trace; and
a plurality of openings, wherein each opening of the plurality of openings is disposed between a respective pair of conductive segments.

19. The cantilever spring of claim 18, wherein the conductive trace comprises conductive polylactic acid.

20. The cantilever spring of claim 18, wherein the cantilever spring is configured to generate an electrical signal in response to a force applied to the cantilever spring.
